(19) United States
(12) Patent Application Publication
(10) Pub. No.: US 2026/0232082 A1
(43) Pub. Date: Aug. 13, 2026

(54) BITE BUCKLE

(71) Applicant: Composite Resources, Inc., Rock Hill, SC (US)

(72) Inventors: Michael James Casella, Fort Mill, SC (US); Kangil Cheon, San Francisco, CA (US); Jesse Arnold Fourt, Menlo Park, CA (US); Christopher Steven Hill, San Francisco, CA (US); Thomas Kai Wucherpfennig, San Francisco, CA (US)

(21) Appl. No.: 19/535,851

(22) Filed: Feb. 10, 2026

Related U.S. Application Data
(60) Provisional application No. 63/756,750, filed on Feb. 10, 2025.

(51) Int. Cl. A44B 11/10; A61B 17/132

(52) U.S. Cl. CPC A44B 11/10; A61B 17/1327

(57) ABSTRACT

A self-activating friction buckle is disclosed. The buckle includes a base and a slider coupled by a prismatic joint that permits longitudinal translation. An object routed through a passage in the base applies a longitudinal force to the slider, causing the slider to move from an open position toward a closed position without manual actuation. As the slider advances, a transverse portion carried by the slider at least partially overlies an opening in the base to reduce an aperture of the passage and create a clamping interface. The clamping force increases with the applied tension. Certain embodiments include a passage geometry that facilitates one-handed routing, a releasable lock that holds the slider open until a threshold force is reached, and structural features that accommodate small-diameter loads. The buckle may be incorporated into straps, tourniquets, and other systems requiring automatic tension-based clamping and may include optional reset, indicator, or friction-enhancing features.

### RELATED APPLICATIONS

This application claims priority to and the benefit under 35 U.S.C. § 119(e) of U.S. Provisional Patent Application No. 63/756,750, filed Feb. 10, 2025, entitled “BITE BUCKLE,” the entirety of which is incorporated herein by reference.

### TECHNICAL FIELD

The present disclosure relates to buckles and strap-retention mechanisms, and more particularly to tension-activated friction buckles.

### BACKGROUND

Conventional strap-tightening and retention mechanisms (e.g., ladder locks, cam buckles, and other manually actuated devices) often require a user to operate a lever, cam, or toothed clamp to secure a strap after tightening. These devices can demand two-handed coordination, may be susceptible to accidental release, and can lose effectiveness when contaminated by mud, sand, blood, or other debris. In time-critical scenarios (e.g., emergency medical use) and field environments, these limitations can complicate or delay achieving a sufficient initial cinch and maintaining tension.

There is therefore a need for improved buckles and strap-retention mechanisms that are capable of automatically activating under strap tension. There is further a need to allow one-handed or simplified routing of a strap. There is a further need for a buckle that reliably operates on small-diameter or irregular loads. There is a further need for a buckle that maintains performance in adverse environmental conditions.

### SUMMARY OF THE DISCLOSURE

Embodiments of the present disclosure relate to self-activating buckles configured to clamp a strap using tension generated within the strap itself. Some embodiments may clamp the strap without manual operation of a lever, tooth, cam, or other dedicated locking mechanism. In various embodiments, a buckle includes a base and a slider coupled by a prismatic joint such that the slider translates longitudinally between an open position and a closed position. The slider carries a clamping structure, such as a transverse plate, that, upon translation into the closed position, moves into at least partial alignment with an opening in the base to reduce an aperture of a strap routing passage and clamp the strap between opposing clamping zones of the base and slider. Strap tension applied to a strap interface region on the slider automatically drives the slider toward the closed position and increases clamping force with increasing strap tension (e.g., proportionally in some embodiments), resulting in a buckle that “bites” harder as strap tension increases.

In certain embodiments, the buckle includes a strap routing passage extending through the base and slider, which may be angled to facilitate threading of the strap from a bottom side toward the top side of the buckle to enable one-handed tightening. Some embodiments incorporate a releasable lock, such as interacting detents and indents on the base and slider, configured to hold the slider in the open position until a threshold tension force is reached. The buckle may further include features such as a visual indicator of the slider state, a reset affordance for returning the slider to the open position, and optional serrated or toothless clamping zones.

The buckle geometry may include a curved contact region on a downwardly extending portion of the base and a forward contact region on the slider, together defining a triangular open area sized to receive a portion of a load or body part when tightened around small-diameter objects. This arrangement allows the buckle to pivot relative to the load while maintaining clamping. In some embodiments, the slider may translate beyond an initial clamping position toward a fully closed position, limited by strap compression, enabling firm clamping across single- and multi-layer strap assemblies.

Methods are provided for threading, tensioning, automatically clamping, and resetting the buckle. According to one embodiment, methods are provided for tightening and clamping a strap using the self-activating friction buckle. Some embodiments may include routing the strap through the buckle, applying tension to translate the slider automatically into the clamping position, and maintaining strap tension without manual engagement of a locking mechanism. The buckle may be reset by releasing strap tension and manually applying a longitudinal force to return the slider to the open position.

The buckles disclosed herein may be integrated into tourniquets, cargo straps, anchor-and-working-strap assemblies, and various medical and industrial systems. In one example, a tourniquet includes a strap, a self-activating friction buckle coupled to an end of the strap, and a tightening mechanism such as a windlass. Pulling the free end of the strap automatically actuates the buckle to clamp the strap around a limb, after which the tightening mechanism may further increase circumferential pressure.

One general aspect of the present disclosure includes a self-activating friction buckle that comprises a base and a slider. The base may define at least a portion of a strap routing passage that extends from a first side to a second side of the base. The slider may coupled to the base at a prismatic joint and may be configured for longitudinal translation between an open position and a closed position. The slider may comprise a transverse portion positioned such that, when the slider moves toward the closed position, the transverse portion at least partially overlies the first opening to reduce an aperture of the strap routing passage, where the aperture is defined between the base and the transverse portion of the slider. The slider is configured to translate toward the closed position in response to a longitudinal force applied to the slider by a strap when the strap is routed through the strap routing passage and the strap is under tension. In some embodiments, the slider is further translatable beyond an initial clamping position toward a fully closed position.

According to one embodiment, the strap routing passage includes an angled region configured to guide a free end of a strap from a lower side toward an upper side of the buckle. The angled region may be inclined toward the front of the buckle moving from the lower side to the upper side.

The base may comprise a frame having opposed longitudinal portions and opposed transverse portions bounding the first opening. The slider may comprise a frame having opposed longitudinal portions and opposed transverse portions bounding a second opening. At least one transverse portion of the slider may extend through the first opening. The strap routing passage may be a multi-part strap routing passage.

The buckle may comprise a releasable lock configured to retain the slider in the open position until a threshold force is applied. The releasable lock may comprise, in some embodiments, comprises interacting detents and indents.

According to one embodiment, a transverse portion of the base defines a first clamping region and a transverse portion of the slider defines a second clamping region positioned opposite the first clamping region of the base when the slider is in the closed position. In some embodiments, at least one of the first or second clamping regions comprises serrations, texturing, or a friction-enhancing surface.

The base may include a downwardly extending portion having a curved contact region. A forward region of the slider and the curved contact region of the base define a generally triangular open area configured to receive a portion of a rounded object.

The buckle may be included with a strap assembly that includes strap having a free end and a second end. In some embodiments, the base is anchored to the second end. The free end of the strap may be routed through the strap routing passage.

Another general aspect of the present disclosure includes a method of clamping a strap with a self-activating friction buckle. The method may include routing a free end of the strap through a strap routing passage of the buckle, pulling the free end to increase tension in the strap, and using the strap tension to translate a slider relative to a base of the buckle so that a transverse plate of the slider reduces an aperture of the strap routing passage and clamps the strap between first and second clamping zones, whereby the strap tension maintains the clamping.

These and other embodiments described herein provide improved initial cinch, maintain tension without external locking devices, permit single-routed and one-handed application, perform reliably even in adverse environmental conditions, and provide increased bite force proportional to strap tension. The features may be implemented independently or in combination to achieve the functionality disclosed.

### BRIEF DESCRIPTION OF THE DRAWINGS

FIG. 1 is a diagrammatic representation of one embodiment of a self-activating friction buckle.

FIG. 2 is a diagrammatic representation of a sectional view of one embodiment of a buckle.

FIGS. 3A and 3B are diagrammatic representations of a releasable lock embodiment with detents holding the slider open until a threshold force is applied.

FIGS. 4A and 4B are diagrammatic representations of sectional views illustrating a strap routing passage, contact regions, and an open area according to one embodiment.

FIGS. 5A-5C are diagrammatic representations of one embodiment of a buckle illustrating one embodiment of the operation of the buckle from open to closed positions and a reset action.

FIG. 6 is a diagrammatic representation of one embodiment of a buckle in a fully closed condition.

FIG. 7 is a diagrammatic representation of one embodiment of a buckle used to tighten a strap around an object and maintain tension.

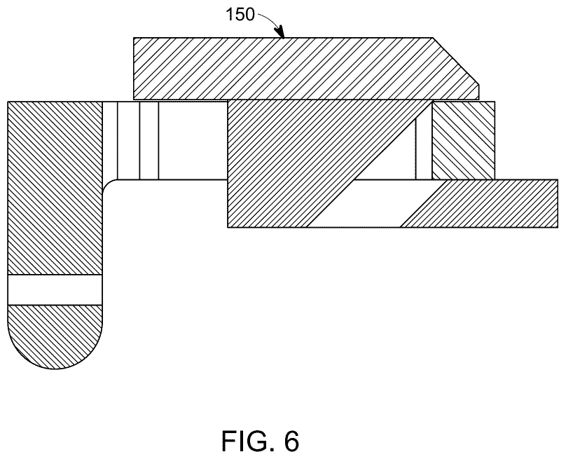

FIG. 8 is a diagrammatic representation of one embodiment of a dual-strap (anchor/working) configuration securing an object with the buckle.

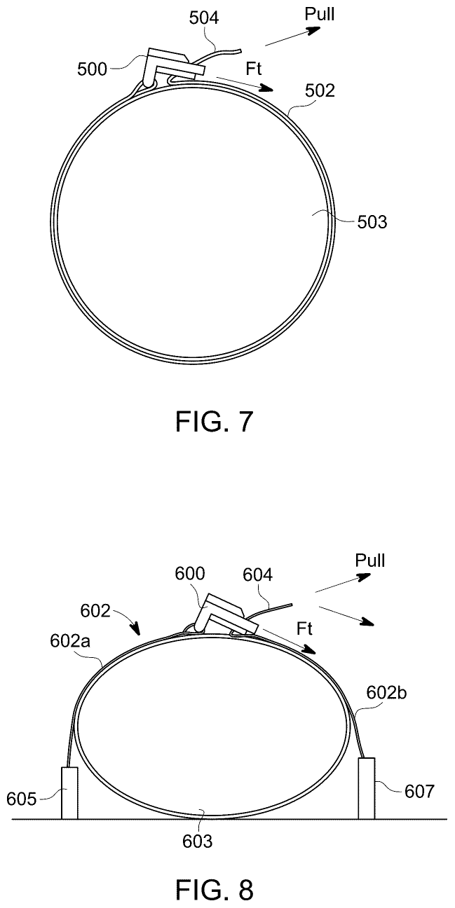

FIG. 9 is a diagrammatic representation of one embodiment of a tourniquet incorporating the buckle, a windlass, and a securing mechanism.

FIG. 10 is a diagrammatic representation of another embodiment of a buckle.

### DETAILED DESCRIPTION

The disclosure and various features and advantageous details thereof are explained more fully with reference to the exemplary, and therefore non-limiting, embodiments illustrated in the accompanying drawings and detailed in the following description. It should be understood, however, that the detailed description and specific examples, while indicating the preferred embodiments, are given by way of illustration only and not by way of limitation. Descriptions of known techniques may be omitted so as not to unnecessarily obscure the disclosure in detail. Various substitutions, modifications, additions and/or rearrangements within the spirit and/or scope of the underlying inventive concept will become apparent to those skilled in the art from this disclosure.

The terms “top,” “bottom,” “front,” “back,” and similar directional references are relative and used herein for descriptive purposes only and are not meant to limit the scope of the invention. Embodiments of the invention can be employed in orientations other than those specifically illustrated.

Embodiments provide a self-activating buckle that uses strap tension to actuate a slider and clamp the strap. In operation, tension in the strap drives a clamping structure (e.g., a transverse plate) to reduce an aperture of a strap routing passage, thereby clamping the strap between opposing clamping zones and maintaining tension without, in some embodiments, a separate manual locking step. The buckle may serve as a primary holding device or work in conjunction with secondary fasteners (e.g., hook-and-loop). In certain embodiments, the buckle includes a strap routing passage extending through the base and slider, which may be angled to facilitate threading of the strap from a bottom side toward the top side of the buckle to enable one-handed tightening.

Methods of using the buckle may include routing a strap through a strap passage, pulling a free end to produce tension, and using the generated tension to automatically actuate the slider into a clamping configuration. The strap may remain clamped as long as some tension is present, including tension from the strap elasticity or load forces, without requiring a secondary locking mechanism. The buckle may be reset to the open position by releasing strap tension and applying an external longitudinal force on the slider.

Some embodiments incorporate a downwardly extending portion of the base having a curved contact region, which cooperates with a forward contact region on the slider to form a generally triangular open area sized to receive a portion of a load or body part when the buckle is tightened against small diameter objects. This geometry enables improved performance on limbs and small cylindrical objects and allows the buckle to pivot relative to the load while maintaining clamping force.

Embodiments may be used in a variety of systems, including tourniquets (e.g., junctional tourniquets, arterial tourniquets), cargo straps, rescue straps, anchor-and-working-strap assemblies, and other medical or industrial devices. In one example, a tourniquet includes a strap with an outer sleeve and an inner tightening strap, a self-activating friction buckle coupled to the strap, and a windlass-type tightening mechanism. Pulling the free end of the strap through the buckle cinches the strap around a limb and automatically clamps the strap, while subsequent windlass rotation increases circumferential pressure. Additional strap-based systems may employ hook-and-loop or other fastening materials to supplement retention if desired.

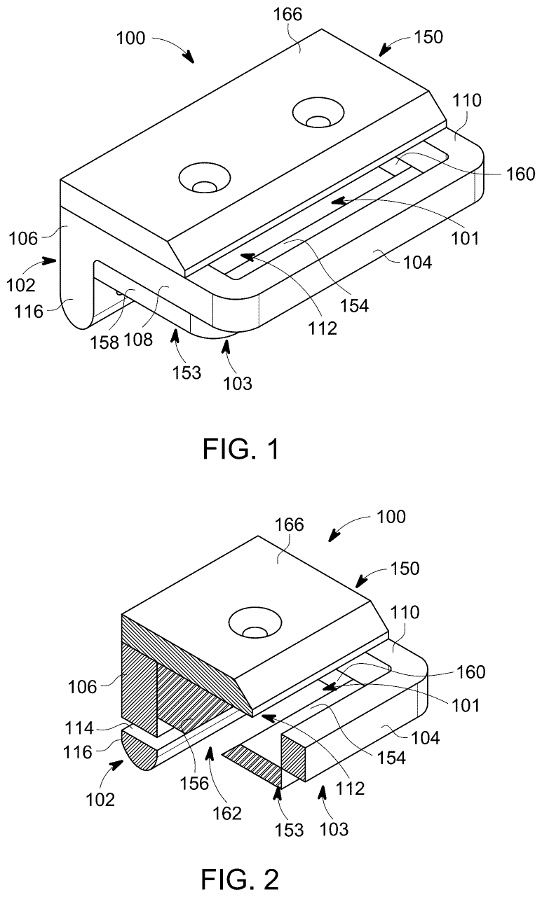

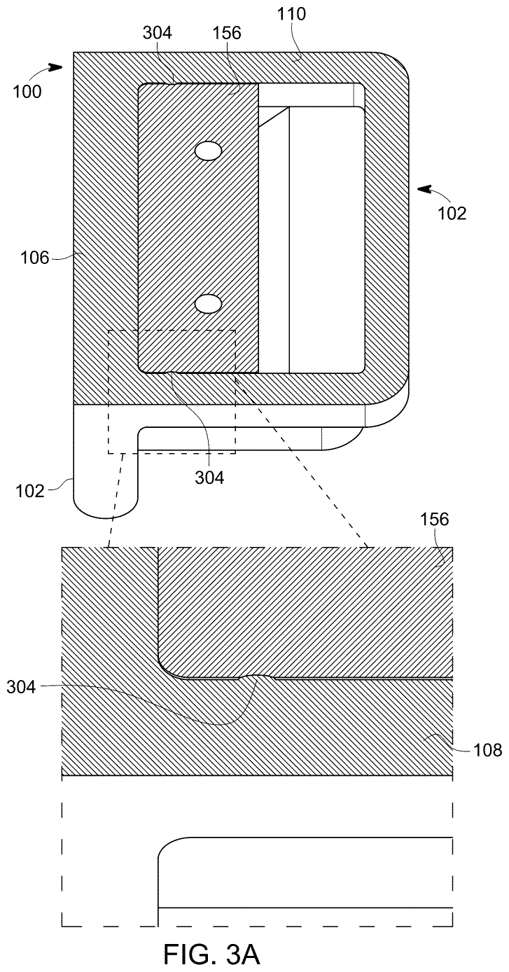

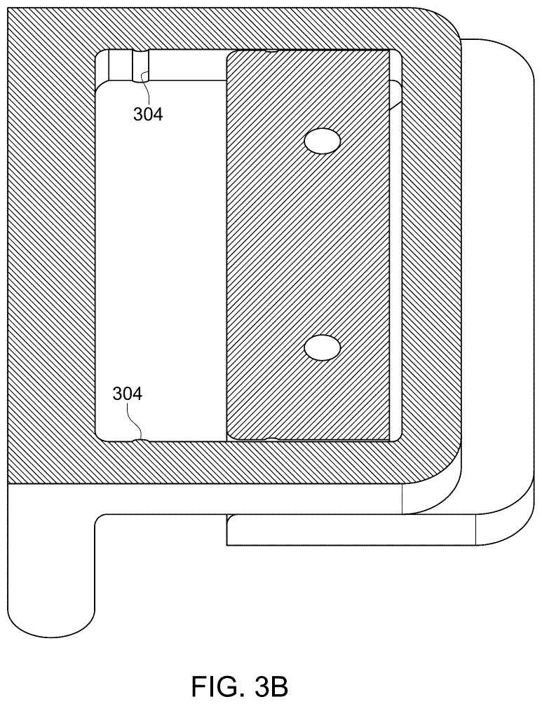

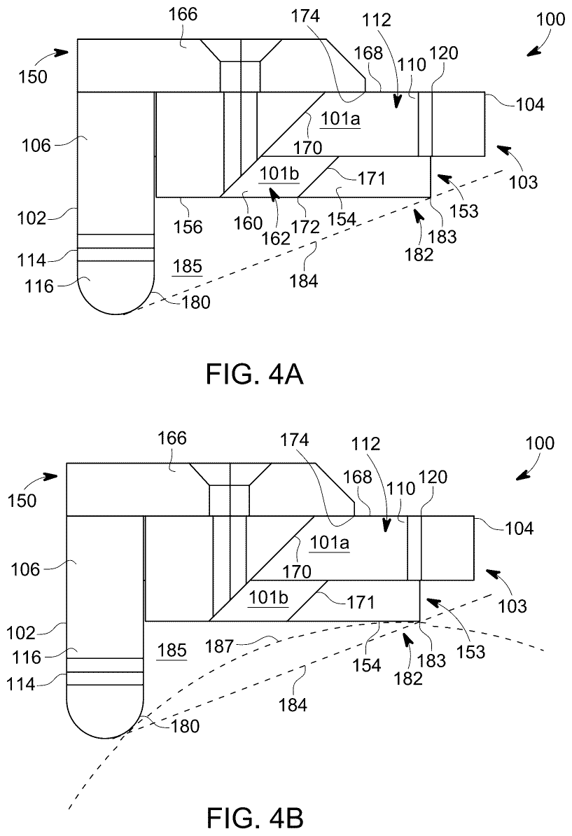

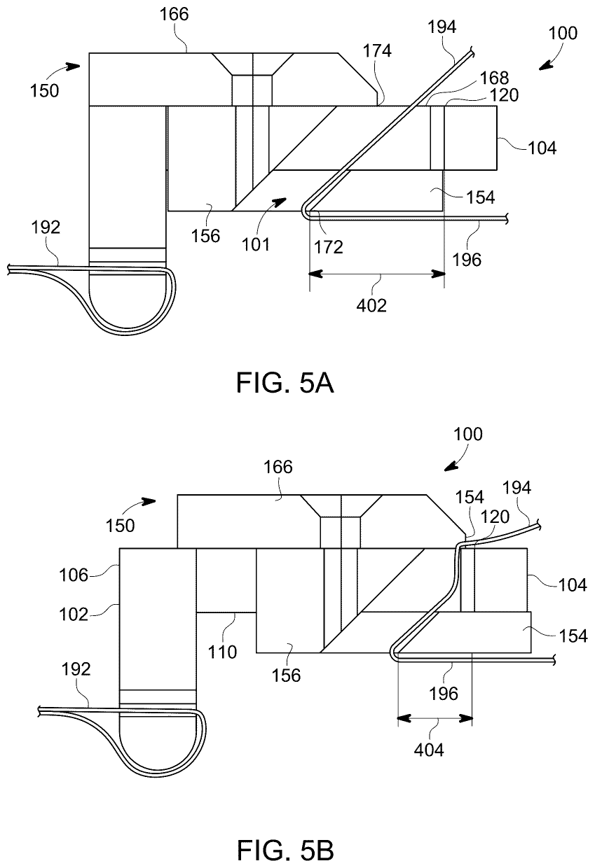

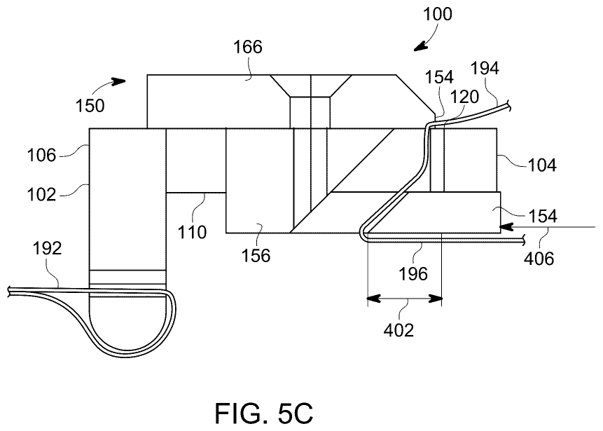

FIG. 1 is a diagrammatic representation of one embodiment of a self-activating friction buckle 100. FIG. 2 is a sectional view of buckle 100. FIG. 3A and FIG. 3B illustrate additional sectional views of one embodiment of buckle 100. FIG. 4A and FIG. 4B illustrate another sectional view of buckle 100. FIG. 5A, FIG. 5B and FIG. 5C illustrate one embodiment of the operation of buckle 100. FIG. 6 illustrates another sectional view of one embodiment of buckle 100.

Buckle 100 comprises a kinematic pair that includes a base 102 and a slider 150 that slides relative to base 102. According to one embodiment, for example, base 102 and slider 150 are connected at a prismatic joint that allows translation of slider 150 relative to base 102 from an open position to a closed position to clamp a strap. A releasable lock feature, such as indents/detents may be used to hold slider 150 in the open position until a threshold amount of force is applied to move slider 150.

Base 102 includes frame 103 having transverse portion 104, transverse portion 106, longitudinal portion 108 and longitudinal portion 110 that bound an opening 112 therethrough from a first side to a second side of frame 103. The longitudinal portions provide lateral sidewalls of opening 112 and the transverse portions provide front and rear sidewalls of opening 112. A downwardly extending portion 116 of base 102 extends downward from transverse portion 106 and includes a transverse slot 114 for attachment of base 102 to a loop of a strap. A rear region of transverse portion 104 provides a clamping zone 120 (FIGS. 4A-4B).

According to one embodiment, slider 150 comprises frame 153 having transverse portion 154, transverse portion 156, longitudinal portion 158 and longitudinal portion 160 that bound a second opening 162 that extends from a first side to a second side of frame 153. Thus, opening 162 is bounded by longitudinal portions and transverse portions of frame 153, with the longitudinal portions providing lateral sidewalls of opening 162 and the transverse portions providing front and rear sidewalls of opening 162. Frame 153 may be superimposed on and slides under frame 103.

Slider 150 further includes a transverse portion 166 (e.g., a plate) that, in the closed position, overlies the first opening 112 to reduce an aperture 168 of the strap routing passage. In certain embodiments, transverse portion 166 slides over opening 162 of frame 153. The rear portion of transverse portion 154 provides a strap interface region 172 and the front area of transverse portion 166 provides a second clamping zone 174. In some embodiments, serrations or other grip-enhancing profiles may be included on clamping zones 174 or 120 to enhance strap retention. The base 102 and slider 150 may form a prismatic joint restricting the slider to longitudinal translation relative to the base.

Buckle 100 forms a strap routing passage 101 through which a strap can be routed from a first side to a second side of buckle 100. More particularly, the base 102 and slider 150 cooperate to form the strap routing passage 101 with portions 101a and 101b, bounded by the frames 103, 153 and transverse portions as shown in FIGS. 2-3. Strap routing passage 101 may be angled to help with the routing while also allowing for enough offset on the bottom moving plate (frame 153) to allow the buckle to engage when tension is applied to the band. In the illustrated embodiment, the rear transverse face 170 and front transverse face 171 are inclined toward a front end of the buckle from bottom to top, to help guide a free strap end for one-handed threading. Routing may proceed from a bottom side to the top side of the buckle. Pulling on the strap to create sufficient tension in the strap causes slider 150 to slide from a first position to second position, closing the aperture of strap routing passage 101 to clamp the strap in place. Continued tension in the strap helps hold slider 150 in the second position.

In the embodiment illustrated, slider 150 includes transverse portion 156 that extends through and slides in opening 112 of frame 103. Thus, as illustrated in FIG. 4A, strap routing passage 101 includes a first portion 101a defined by transverse portion 104, transverse portion 156, and the longitudinal portions 108, 110 of frame 103 and a second portion 101b defined by transverse portion 154, transverse portion 156, and the longitudinal portions 158, 160 of frame 153. The rear transverse face 170 of strap routing passage 101 and the front transverse face 171 are angled toward the front end of buckle 100 moving from bottom to top. These angles can help guide the free end of a strap as it is threaded through buckle 100. In particular, the angled path allows for one-handed tightening of a strap around a range of size of objects.

With reference to FIG. 4A, portion 116 of base 102 extends downward past the lower face of frame 153 and includes a first contact region 180 that contacts or otherwise compresses a strap (e.g., a working strap or an anchor strap) against the load being compressed or secured using the strap. Contact region 180 is curved to allow buckle 100 to pivot. Further, a forward area of slider 150 (for example, a region of the bottom surfaces of one or more of longitudinal portions 158, 160, or the leading edge 183 of frame 153) forms a second contact region 182 that contacts or otherwise compresses a strap (e.g., a working strap or an anchor strap) against the load being compressed or secured using the strap. Together with a tangent plane 184, these features define a generally triangular open area 185 into which a portion of a small-diameter or contoured load can project (e.g., a limb), enabling secure clamping and buckle pivoting while maintaining bite force.

In the embodiment illustrated, open area 185 has a generally tilted right-triangle cross-sectional shape formed by a bottom surface plane of frame 153, a front surface plane of portion 116 of base 102 and an imaginary plane 184 tangent to first contact region 180 and leading edge 183 to form an open area 185. As illustrated in FIG. 4B, if buckle 100 is tightened against the outer surface of a generally circular or elliptical load (represented by line 187), a portion of the load can project into open area 185. The offset distance between contact area 180 and contact region 182, and the triangular empty space between, help achieve bite force on small diameter objects. In a tourniquet application, for example, open area 185 may be sized such that the limb of a small child may be at least partially received in open area 185 to allow the tourniquet to be fully cinched about the child's arm.

In one embodiment, buckle 100 includes a releasable lock to maintain buckle 100 in an open position until a threshold amount of force is applied to move slider 150 relative to base 102. Examples of releasable lock features include, but are not limited to, indents and detents. In the embodiment of FIG. 3A, transverse portion 156 of slider 150 includes indents and longitudinal portions 108, 110 of frame 103 include detents 304 that mate with the indents of transverse portion 156. When a desired threshold amount of force is applied, the material of buckle 100 deforms, allowing slider 150 to slide longitudinally relative to frame 103 to a closed position (FIG. 3B). The lock may provide audible/tactile feedback. In some embodiments, slider 150 can be returned to an open and locked position after use.

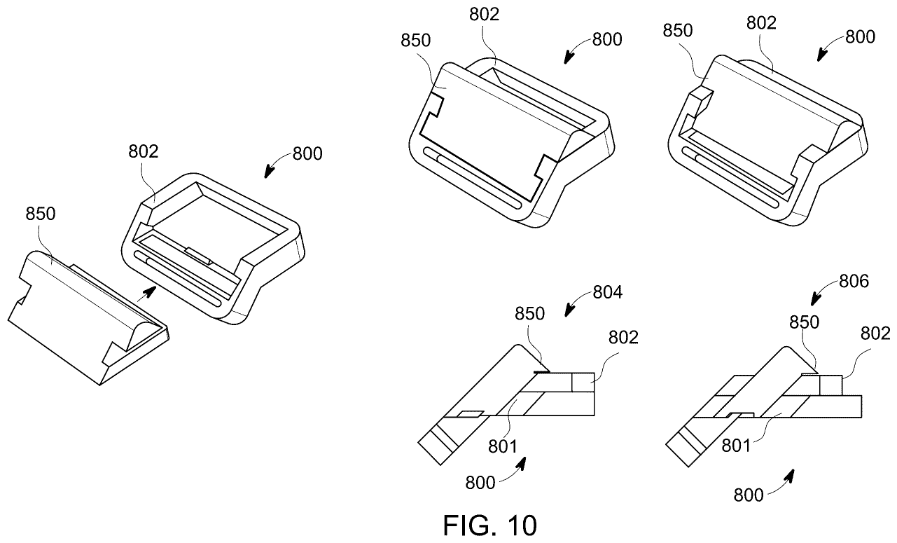

The base 102 and slider 150 may be formed of metals, composites, plastics, or combinations thereof. Embodiments may be toothless (smooth clamping zones) or include serrations/teeth and other surface profiles to enhance grip. In some embodiments, one or more clamping zones (e.g., clamping zone clamping zone 174, clamping zone 120) include teeth or other features. FIG. 10 illustrates a two-part plastic construction that snaps together, includes a visual indicator communicating open/closed state, and the top-side affordance to help open/reset.

In operation (FIGS. 5A-5B), a strap loop 192 or other mechanism couples the base 102 to a strap, and a free end 194 is routed through passage 101 from bottom-to-top. Free end 194 and loop 192 may be portions of the same strap or portions of different straps that act together as a strap. Loop 192 may be a fixed loop or an adjustable loop. The user pulls free end 194 to cinch the strap. Pulling the free end 194 increases strap tension, which applies a longitudinal force Ft to the slider via the strap interface region 172. As tension in the strap increases, the strap asserts a longitudinal force on transverse portion 154 (e.g., at strap interface region 172). A sufficient longitudinal force on transverse portion 154 causes slider 150 to slide longitudinally relative to base 102. As such, slider 150 moves from an open position (with an initial offset 402) toward a closed clamping position (with a reduced offset 404) and transverse portion 166 reduces aperture 168 to clamp the strap between clamping zones 120 and 174. According to one embodiment, buckle 100 bites into strap 196 with a force that is proportional to the band's tension. As tension in the strap increases, the biting force of buckle 100 also increases.

Continued tension in the working portion of the strap 196—such as tension from the elasticity of the strap, a load pulling on strap, etc.—will continue to result in a longitudinal force Ft on transverse portion 154, thus helping to maintain the clamping force of buckle 100 on the strap. As such, buckle 100 maintains the tension in the strap.

Some embodiments include additional mechanisms to help maintain tension in the strap. For example, the outer surface of the strap may include one or more lengths of hook and loop material so that when free end 194 of the strap is looped through buckle 100, the outer surface of the strap may be applied to itself, thereby helping to secure the position of the strap. In addition, or in the alternative, to using hook and loop material as a fastener, the strap may be fitted with other types of fasteners, such as, but not limited to rigid hooks and loops, buttons, snaps, transverse straps, or other fastening mechanisms.

Thus, buckle 100 is a self-activating friction buckle that uses the tension of the strap to activate the buckle. Once activated, buckle 100 is able to hold the strap tension, either alone, or in conjunction with additional mechanisms. With reference to FIG. 5C, buckle 100 can be reset by releasing tension in strap 196 and applying a longitudinal force 406 to slider 150 to move slider 150 back to an open position.

In some embodiments, such as illustrated in FIG. 6, slider 150 can move past the point required to bite into the strap and, but for the obstruction of the strap, fully close aperture 168. That is, slider 150 can translate beyond the initial clamping point toward a fully closed position, such that the strap itself limits closure. This behavior helps clamp and hold tension in multi-layer straps, such as tourniquet straps that have a sleeve/inner strap arrangement in which the inner strap can slide relative to the outer sleeve. Slider 150 can continually close until the strap can no longer compress, thus holding tension in both the inner band and the outer band.

FIG. 5 is a diagrammatic representation of one embodiment of a self-activating friction buckle 500 used to tighten a strap 502 about an object 503. In the embodiment of FIG. 5, buckle 500 is attached to working strap 502 using a fixed loop, an adjustable loop, or another mechanism. The free end 504 of strap 502 is routed through buckle 500. Pulling free end 504 cinches strap 502 about object 503 and causes buckle 500 to activate to bite (clamp) and hold strap 502. When pulled sufficiently tight, tension in strap 502 (Ft) helps maintain the bite even after the user has stopped pulling free end 504.

Some embodiments include additional mechanisms to help maintain tension in strap 502. For example, the outer surface of strap 502 may include one or more lengths of hook and loop material so that when the free end 504 of strap 502 is looped through buckle 500, the outer surface of strap 502 may be applied to itself, thereby helping to secure the position of strap 502. In addition, or in the alternative, to using hook and loop material as a fastener, strap 502 may be fitted with other types of fasteners, such as, but not limited to rigid hooks and loops, buttons, snaps, transverse straps, or other fastening mechanisms.

FIG. 6 is a diagrammatic representation of another embodiment of a self-activating friction buckle 600 used to tighten a strap 602 to secure an object 603. In this example, strap 602 includes an anchor strap 602a and a working strap 602b. In the embodiment of FIG. 6, buckle 600 is attached to an anchor strap 602a using a fixed loop, an adjustable loop, or another mechanism. The fixed end of anchor strap 602a is anchored to structure 605 (e.g., using a fixed loop, an adjustable loop, a hook, or other mechanism). Similarly, a fixed end of working strap 602b is anchored to a structure 607 (e.g., using a fixed loop, an adjustable loop, a hook, or other mechanism), which may be the same structure as structure 605 or a different structure. The free end 604 of working strap 602b is routed through buckle 600. Pulling free end 604 tightens strap 602 about object 603 and causes buckle 600 to activate to bite (clamp) and hold strap 602. When pulled sufficiently tight, tension in strap 602 (Ft)—more particularly, tension in working strap 602b, helps maintain the bite, and thus strap tension, even after the user has stopped pulling free end 604.

Some embodiments include additional mechanisms to help maintain tension in strap 602. For example, the outer surface of strap 602b may include one or more lengths of hook and loop material so that when free end 604 of strap 602b is looped through buckle 600, the outer surface of strap 602b may be applied to itself, thereby helping to secure the position of strap 602b. In addition, or in the alternative, to using hook and loop material as a fastener, strap 602b may be fitted with other types of fasteners, such as, but not limited to rigid hooks and loops, buttons, snaps, transverse straps, or other fastening mechanisms.

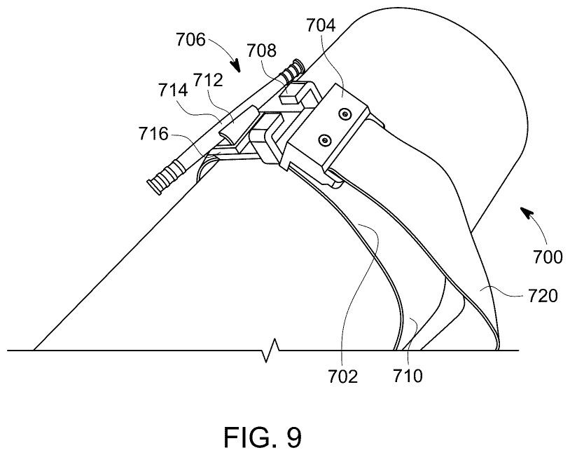

Embodiments of self-activating friction buckles of the present disclosure may be used for a variety of applications. FIG. 9, for example, is a diagrammatic representation of one embodiment of tourniquet 700, which comprises a tourniquet strap 702, self-activating friction buckle 704, a tightening mechanism 706, and a securing mechanism 708.

Tourniquet strap 702 includes an outer sleeve 710 and an inner strap 712. Tightening mechanism 706 is used to tighten the inner strap 712. According to one embodiment, tightening mechanism 706 comprises a windlass 714 and a strap guide 716 to route the inner tightening strap 712 to/from windlass 714 from/to the interior area of outer sleeve 710. Securing mechanism 708 includes features to prevent windlass 714 from unwinding.

According to one embodiment, buckle 704 is attached to one end of tourniquet strap 702 using a fixed loop, an adjustable loop, or another mechanism. Free end 720 of tourniquet strap 702 is routed through buckle 704 to form a loop. When tourniquet 700 is applied to a limb, the user pulls end 720 to cinch strap 702 about the limb. Cinching strap 702 causes buckle 704 to activate to bite (clamp) and hold strap 702. Tension in strap 702 helps maintain the bite even after the user has stopped pulling free end 720. Thus, buckle 704 helps maintain strap tension during use.

Some embodiments include additional mechanisms to help maintain tension in strap 702. For example, the outer surface of strap 702 may include one or more lengths of hook and loop material so that when the free end of strap 702 is looped through buckle 704, the outer surface of strap 702 may be applied to itself, thereby helping to secure the position of strap 702. In addition, or in the alternative, to using hook and loop material as a fastener, strap 702 may be fitted with other types of fasteners, such as, but not limited to rigid hooks and loops, buttons, snaps, transverse straps, or other fastening mechanisms.

Once the initial cinch is achieved, the user winds windlass 714 to tighten inner strap 712, thereby causing the tourniquet to apply circumferential pressure to the body part. When sufficient pressure is achieved, the user hooks the end of the windlass 714 under a hooked catch of securing mechanism 708. A clip, transverse strap, or other mechanism may be used to retain windlass 714 in securing mechanism 708.

FIG. 10 illustrates another embodiment of a self-activating friction buckle. The buckle of FIG. 10 is designed with a two-part construction in plastic that snaps together. In this example, the buckle 800 comprises a base 802 and a slider 850. Slider 850 can be inserted in base 802 so that the frame of slider 850 is superimposed on and slidable under the frame of base 802. Base 802 includes a slot to allow base 802 to be attached to the loop of a strap. The working end of a strap may be routed through the strap routing passage 801 of buckle 800. Pulling on the strap to create sufficient tension in the strap causes slider 850 to slide from a first position 804 to a second position 806, closing the aperture of the strap routing passage and clamping the strap in place. Continued tension in the strap helps hold slider 850 in the second position. The buckle 800 has a visual indicator to communicate its state (open vs. closed), and it has an affordance on the top to help the user open and reset the buckle.

Buckles according to the present disclosure can provide a number of advantages when incorporated into tourniquet systems. In various embodiments, the buckle enables an improved initial cinch, allowing the user to quickly establish baseline strap tension during application. After the initial cinch is achieved, the strap tail may be pulled again to further tighten the strap without releasing or disrupting the tension already applied. The buckle may also enable one-handed, single-route application, thereby reducing application time and simplifying operation in both routine and high-stress environments.

In some embodiments, the buckle is capable of maintaining pressure without relying on supplemental holding mechanisms such as hook-and-loop materials. When such secondary mechanisms are used in combination with the buckle, they may provide beneficial redundancy and additional security. The buckle can also exhibit improved performance in adverse environmental conditions including mud, sand, moisture, and blood where hook-and-loop fasteners may become less reliable.

The structure of the buckle enables a bite force that increases (e.g., proportionally) with strap tension and is capable of retaining and preventing slippage of an inner strap. In addition, the buckle can generate sufficient bite force to securely engage small-diameter objects, including pediatric limbs or compact cylindrical structures. In certain embodiments, features such as detents or similar mechanisms may be incorporated to hold the jaws open for easier application and, if desired, to provide audible or tactile feedback upon engagement.

It will be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the meaning of “in” includes “in” and “on” unless the context clearly dictates otherwise.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” “in one embodiment.”

Thus, while the invention has been described with respect to specific embodiments thereof, these embodiments are merely illustrative, and not restrictive of the invention. Rather, the description is intended to describe illustrative embodiments, features and functions in order to provide a person of ordinary skill in the art context to understand the invention without limiting the invention to any particularly described embodiment, feature or function, including any such embodiment feature or function described. While specific embodiments of, and examples for, the invention are described herein for illustrative purposes only, various equivalent modifications are possible within the spirit and scope of the invention, as those skilled in the relevant art will recognize and appreciate.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component.

What is claimed is:

1. A self-activating friction buckle, comprising:
a base defining at least a portion of a strap routing passage extending from a first side to a second side of the base; and
a slider coupled to the base by a prismatic joint and configured for longitudinal translation between an open position and a closed position, wherein the slider comprises a transverse portion positioned such that, when the slider moves toward the closed position, the transverse portion at least partially overlies the first opening to reduce an aperture of the strap routing passage, wherein the aperture is defined between the base and the transverse portion of the slider, wherein the slider is configured to translate toward the closed position in response to a longitudinal force applied to the slider by a strap when the strap is routed through the strap routing passage and the strap is under tension.

2. The buckle of claim 1, wherein the strap routing passage includes an angled region configured to guide a free end of a strap from a lower side toward an upper side of the buckle.

3. The buckle of claim 1, wherein the base comprises a frame having opposed longitudinal portions and opposed transverse portions bounding the first opening, and the slider comprises a frame having opposed longitudinal portions and opposed transverse portions bounding a second opening, wherein a transverse portion of the slider extending through the first opening, wherein the strap routing passage is a multi-part strap routing passage.

4. The buckle of claim 1, further comprising a releasable lock configured to retain the slider in the open position until a threshold force is applied.

5. The buckle of claim 4, wherein the releasable lock comprises indents on the slider and detents on the base.

6. The buckle of claim 1, wherein the base defines a first clamping region and the transverse portion of the slider defines a second clamping region positioned opposite the first clamping region of the base when the slider is in the closed position.

7. The buckle of claim 6, wherein at least one of the first or second clamping regions comprises serrations, texturing, or a friction-enhancing surface.

8. The buckle of claim 1, wherein the base includes a downwardly extending portion having a curved contact region.

9. The buckle of claim 8, wherein a forward region of the slider and the curved contact region of the base define a generally triangular open area configured to receive a portion of a rounded object.

10. The buckle of claim 1, wherein the slider is further translatable beyond an initial clamping position toward a fully closed position.

11. A strap assembly, comprising:
a strap having a free end and a second end; and
a self-activating friction buckle comprising:
a base anchored to the second of the strap, the base defining at least a portion of a strap routing passage for routing a free end of the strap through the self-activating frication buckle, the at least the portion of the strap routing passage extending from a first side to a second side of the base, wherein the base defines a first clamping region; and
a slider coupled to the base by a prismatic joint and configured for longitudinal translation between an open position and a closed position, wherein the slider comprises a transverse portion positioned such that, when the slider moves toward the closed position, the transverse portion at least partially overlies the first opening to reduce an aperture of the strap routing passage, wherein the slider comprises a second clamping region, wherein the aperture is defined between the first clamping region and the second clamping region, wherein the slider is adapted to translate toward the closed position in response to a longitudinal force applied to the slider by the strap when the strap is routed through the strap routing passage and is under tension to clamp the strap between the first clamping region and the second clamping region.

12. The strap assembly of claim 11, wherein the passage is angled to facilitate one-handed routing of the strap.

13. The strap assembly of claim 11, wherein the strap includes an outer sleeve and an inner tightening band.

14. The strap assembly of claim 11, wherein the buckle clamps multiple layers of the strap until compressive limits of the layers are reached.

15. The strap assembly of claim 11, wherein the strap includes hook-and-loop material or a mechanical fastener configured to secure the free end after clamping.

16. The strap assembly of claim 11, wherein a clamping force applied by the buckle to the strap increases proportionally with tension in the strap.

17. The strap assembly of claim 11, wherein the base comprises a frame having opposed longitudinal portions and opposed transverse portions bounding the first opening, and the slider comprises a frame having opposed longitudinal portions and opposed transverse portions bounding a second opening, wherein a transverse portion of the slider extends through the first opening, wherein the strap routing passage is a multi-part strap routing passage.

18. The strap assembly of claim 11, further comprising a releasable lock configured to retain the slider in the open position until a threshold force is applied.

19. The strap assembly of claim 18, wherein the releasable lock comprises indents on the slider and detents on the base.

20. The strap assembly of claim 11, wherein the base includes a downwardly extending portion having a curved contact region.

21. The strap assembly of claim 11, wherein a forward region of the slider and the curved contact region of the base define a generally triangular open area configured to receive a portion of a rounded object.

22. A method of clamping a strap with a self-activating friction buckle, comprising:
routing a free end of the strap through a strap routing passage of the buckle;
pulling the free end to increase tension in the strap; and
using the strap tension to translate a slider relative to a base of the buckle so that a transverse plate of the slider reduces an aperture of the strap routing passage and clamps the strap between first and second clamping zones, whereby the strap tension maintains the clamping.
